(19) United States
(12) Patent Application Publication
(10) Pub. No.: US 2008/0077432 A1
(43) Pub. Date: Mar. 27, 2008

(54) Comprehensive tissue management system

(76) Inventor: Duke O. Kasprisin, South Burlington, VT (US)

Correspondence Address: Moss & Barnett P.A.;4800 Wells Fargo Center, 90 South Seventh Street, Minneapolis, MN, 55402-4129, US

(21) Appl. No.: 11/540,844

(22) Filed: Sep. 29, 2006

Related U.S. Application Data
(60) Provisional application No. 60/826,492, filed on Sep. 21, 2006.

(51) Int. Cl. G06Q 10/00; A61B 5/00

(52) U.S. Cl. 705/2; 600/300

(57) ABSTRACT

The present invention provides a comprehensive tissue management method and system for transplantable materials like tissues and organs. The tracking portion of the system verifies that staff members of a medical establishment like a hospital have handled, stored, transported, reconstituted, and used the tissue or organ materials in a safe and regulatory-compliant manner from the point of receipt to the point of issuance or surgical use throughout the hospital's organization. The tracing portion of the system creates an integral record that documents which hospital staff members have provided which processing steps to the tissue or organ, any associated materials used in conjunction with such tissue or organ, and an identification of the tissue or organ that was transplanted or implanted inside a patient. Such a system will enable adverse reaction investigations for transplant patients.

### CROSS-REFERENCE TO RELATED APPLICATION

This application claims the benefit of the U.S. provisional application No. 60/826,492 entitled “Tissue Tracking and Tracing System” filed on Sep. 21, 2006, which is hereby incorporated by reference.

### FIELD OF THE INVENTION

This invention relates generally to transplantable materials including cells, tissues and organ transplants (“TM”) within the medical field, and more specifically to a system for ensuring the safe and appropriate handling of such TM from receipt to their use in surgical procedures and tracing such TM later in response to an adverse reaction investigation.

### BACKGROUND OF THE INVENTION

The human body is made up of trillions of cells that allow it to function, grow, heal and defend itself against hundreds of diseases. Cells of the same type combine to form tissues. Examples of tissues include: connective tissue which helps to support and join together various parts of the body; epithelial tissue which acts as a covering for external and internal body surfaces; muscle tissue which consists of threadlike fibers that can contract to make movement of the body possible; and nervous tissue which carries signals to permit various parts of the body to communicate with each other.

An organ consists of multiple tissues working to perform a particular function. For example, connective, epithelial, muscle and nervous tissues all combine to make up the heart which pumps blood throughout the body. The body's skin, skeletal, muscular, digestive, respiratory, circulatory, urinary, lymphatic, endocrine and nervous systems are all formed from multiple organs that in turn are made up of several different kinds of tissues.

When tissues or organs are damaged or diseased, healthy cells, tissues, or organs from one person may be transplanted to replace the diseased, damaged, or destroyed tissue or organ in another person. Blood transfusion is the most common type of tissue transplanted. After blood products the most common transplant from person (allogeneic) is cadaveric tissue: e.g., bone, tendons, skin, cornea, heart valves and blood vessels. There are more than a million tissue transplants a year; most of these are bone and other musculoskeletal tissues. For instance, bone transplants are commonly used in spinal surgery and the transplanted bone attracts new bone formation and eventually becomes an indistinguishable part of the recipient's bone. Cornea transplants improve the vision of the patients whose corneas have become scarred by injury or clouded by age or infection. Skin transplants can be used to temporarily cover areas of the body of burn victims to reduce the risk of infection, prevent the loss of fluids and decrease pain until skin from another part of the patients own body can be utilized for a more permanent transplant. Bone marrow transplants replace the blood-forming tissue within a patient's bones to treat certain kinds of cancer and serious blood disorders.

Tissues are usually isolated and processed for easier use in surgical procedures. For example, whole bones can be used in transplant or bones can be cut into various shapes or powdered to use in filling voids. Bones from different areas of the body can be combined and processed by proprietary methods into new products designed for unique uses in surgical procedures.

Some tissues can be treated more harshly than others and the processing methods will determine the likelihood that a tissue product carries an infectious risk. Tissues such as bone that are treated with alcohol, oxidative agent or irradiation have very low or non-existent risks, but some surgeons are reluctant to use these for fear that the functionality of the product has been compromised. Use of antibiotics does not guarantee that bacteria have been totally removed from the graft and have no effect on viruses.

The heart, lungs, kidney and liver are commonly transplanted organs. Such transplant procedures can enhance the quality of life for some patients and restore the health of people who may otherwise die, Some organs like the heart and lungs cannot survive outside the body for more than a couple of hours. Thus, they usually are transplanted quickly from a patient who has been declared brain dead and surviving only by mechanical means in order to perfuse the organs until they can be transplanted to the matched recipient patient in need. Organ registries have been established in many cities and countries to identify and prioritize transplant patients in need of an organ. Their position on the registry list will typically determine, when, if ever, they can qualify for a transplant. Many tissues like bone, corneas and skin, however, can be held for longer periods outside the human body. Such tissues can be stored for future transplantation or implantation in refrigerators or freezers at independent tissue banks or tissue processors and subsequently distributed to storage units within hospitals.

Infection from contaminated grafts is the greatest risk from transplantation. Tissue and organ transplantations have been associated with risk from HIV, hepatitis, bacterial infections, prion associated diseases such as Creutzfeldt-Jakob Disease (CJD), rabies, fungus infections, West Nile virus, leucocytic choriomeningitis, as well as many others. Any transplant operation carries the risk of rejection by the body's immune system or infection. Surgeons try to prevent rejection by choosing a donor with the same blood type as the recipient patient. Matching HLA antigens between the donor and recipient may also be important for kidney and bone marrow transplants. Immuno-suppressive drugs like azathioprine, prednisone and cyclosporine are also commonly given to the transplant patient to help prevent tissue or organ rejection. Because such immuno-suppressive drugs act to reduce immune activity within the patient, they may also hinder the body's ability to defend itself against infections.

Improper handling of the cells, tissues or organs prior to its use in the transplant operation can adversely impact its functionality once implanted in the patient, or greatly increase the likelihood of an infection or other adverse reaction by the patient. For example, the TM may be stored at the incorrect temperature or outside a sterile environment. The packaging surrounding the TM may become perforated. Prior to surgery, TM may be improperly reconstituted. Blood and tissue banks are typically better than surgical units in hospitals at establishing some procedures for storing cells, tissues and organs; however, once these materials leave their facilities, the safety system can deteriorate rapidly. Hospitals rarely have established policies and procedures for receiving, handling, storing and reconstituting tissues and organs before their use in surgery. Instead, they allow a great deal of individual discretion to the hospital physicians and nurses for these critical activities. Consequently, standards and procedures can differ greatly across the hospital staff to the potential detriment of the patient.

Many hospitals perform no qualification of the donor sources of tissue and organs that they use in surgical procedures. To the extent that the hospitals institute any certification process for their tissue and organ suppliers, the process tends to be directed to issues of price and delivery schedule, instead of whether the supplier is properly registered, licensed, and compliant with prevailing industry safety standards. It is as if tissue and organs are just another form of paper clips that need to be stocked in the hospital's inventory. Suppliers of tissue have even been known on occasion to bring these critical tissues in their car trunks to the hospital operating room without monitoring storage conditions.

While hospital surgical departments may possess refrigeration units for storing tissue and organs, their staffs frequently do not know how to monitor and control the equipment. Moreover, few surgical units possess the necessary training to reconstitute tissue. The blood bank and surgical units within the hospital may possess individual staff members with knowledge but they are outside of each other's control.

All of these problems can lead to adverse reactions, including serious infections, illnesses and even death for the transplant patient. When such an incident occurs, good medical practice and public health policy requires an immediate investigation of the patient's condition to determine whether the infection, illness or death was caused by the tissue, organ, or surgical procedure, as opposed to an independent condition in the patient. If the surgical procedure was faulty, then the transplant procedure needs to be traced back to the surgeons and nurses involved, the operating room environment, and the equipment involved to reduce the likelihood of a repeat event. If the TM was the cause, then it needs to be traced back to the donor or supplier so that other TM from the same source is immediately removed from inventory and other patients who are transplant recipients of similar TM from the same supplier or donor can be warned and provided appropriate medical care and counseling. Yet, such a tracing process is frequently impossible because many hospitals fail to log in the TM that they receive from suppliers and track their use in surgical procedures. Quality problems in hospitals culminated in 2005 when there was a major recall of tissue products inappropriately released by several tissue banks. Yet, repeated attempts to locate tissue products at hospitals that had not been transplanted failed miserably, thereby resulting in other patients receiving potentially contaminated tissue products. During the same recall, hospital protocols for tracing recipients of the potentially contaminated products were found to be substantially inadequate or entirely absent. Close to one year later, there are recipients yet to be identified who have not received appropriate diagnostic treatments and modalities.

Likewise, most hospitals do not have good systems implemented for gauging compatibility between donors and recipients for organs. In one incident reported within the industry, organs provided by a donor institution resulted in several cases of hepatitis C in the transplanted patients. Because the hospital failed to notify the tissue bank for 16 months, other infected patients were deprived of treatment while this disease could be treated, resulting in additional deaths. A $32 million damage award resulted from a subsequently filed litigation.

In addition, there have been reported cases of physicians taking diseased tissue from in-hospital patients and transplanting it into unsuspecting, healthy patients. These tissues have not been able to be tracked back to the original source, resulting in the recipient's death.

The transplant industry relies upon “tissue usage information cards” that a hospital is supposed to return to the issuing tissue bank after a surgical procedure is completed. Such cards allow the tissue bank to monitor usage of their tissue and notify everyone who has received similar tissue for recall purposes. However, hospitals only return 50-85% of these cards to the tissue banks.

In the case of adverse reaction investigations, hospitals do not usually define what constitutes an adverse reaction and therefore what should be reported. Instead, the reporting responsibility is left to the physician's discretion. Physicians often resort to a gram stain test or cultures on the tissue prior to surgery. But, such test results can be misleading and grossly inadequate to detect diseased tissues.

There are associated risks with tissue transplantation. There are numerous reports of transplant-transmitted infections, including some that resulted in death. For example, there was a recent article (Morbidity and Mortality Weekly Report 2002 (“MMWR”); 51 (March 15^{th}): 207-210) that reported that on Nov. 7, 2001, a 23-year-old male from St. Cloud, Minn. had knee surgery using a refrigerated “fresh” femoral condyle. On Nov. 10, 2001, the patient developed knee pain and severe hypotension. On Nov. 11, 2001, the patient died from clostridium sordelli sepsis that came from the tissue transplant. On Nov. 13, 2002, a 17-year-old male in Illinois also received a “fresh” femoral condyle and meniscus from the same donor. On Nov. 14, 2002, the patient developed a fever and septic arthritis. The presumed cause was a clostridia infection. Likewise, usage of antibiotics in patients prior to surgery can mask problems contained in tissues.

Because of these abuses and other safety problems within hospital and tissue bank environments, regulatory and standard setting agents like the Joint Commission for the Accreditation of Health Care Organization (“JCAHO”), American Association of Blood Banking (“AABB”), Food & Drug Administration (“FDA”), and the College of American Pathologists (“CAP”) are currently implementing mandates for the safe handling, storage, use, and tracing of TM. However, these mandates provide no instructions or guidelines to the hospital or tissue bank for how to comply. Therefore, such hospitals and tissue banks are left with regulatory and legal liability for their failure to comply, but no tools to use to comply.

Some instances of tissue tracking are reported in the prior art. Thus, U.S. Published Application No. 2005/0262088 filed by Solis et al. discloses a system for organ procurement and transfer. While this system maintains the security of patient information, it does not address the safety of the organ or organ match for the patient.

U.S. Published Application Nos. 2005/0010437 and 2005/0010449 filed by Abukwedar teaches an organ donation system that permits a person to donate or agree to donate one type of organ in order to be accorded preferential receipt of another organ. This exchange program, however, does nothing for tracking the safe receipt, handling, or use of the organ, or tracing its use after surgery.

U.S. Published Application No. 2005/0285715 filed by Comunale discloses a container with an electronic lock controlled by a computer system for carrying blood samples or organs to a hospital in a secure manner. While this transportation container can prevent theft or contamination of the organ by strangers, it does nothing to prevent unsafe handling, storage, or treatment by the hospital of the organ.

Other prior art systems exist within a diagnostic laboratory, for tracking biological samples. Thus, U.S. Published Application No. 2003/0120633 filed by Torre-Bueno assigns unique bar codes to samples that can be scanned and read during processing of the sample within the lab. U.S. Pat. No. 5,416,029 issued to Miller et al. employs color-coded embedding media and corresponding color-coded slides accompanied by words, numbers, or symbols to identify the biological samples. U.S. Pat. No. 5,842,179 issued to Beavers et al. discloses a cryogenic freezer with a security key pad for receiving and tracking information to identify the location of blood and tissue samples stored within the freezer, and when a capsule has been removed from the freezer. While these types of systems may be useful for keeping track of thousands of biological samples stored within a diagnostic laboratory, they do nothing to ensure the safe handling, storage, and treatment of the sample within the lab.

Other prior art references disclose systems for keeping track of the whereabouts of surgical supplies used during surgery to detect if they have been accidentally left inside the patient after surgery. See, e.g., U.S. Published Application No. 2002/0049650 filed by Reff, and U.S. Pat. No. 6,861,954 issued to Levin. U.S. Published Application No. 2002/0082957 filed by Krassi specifies an inventory control system for chemical reagents used within a clinical or diagnostic lab. Such inventory control tracking systems can detect the location or number of products, but once again, they do not address the proper handling and storage of those products.

A comprehensive system for tracking the appropriate handling, storage and use of tissues and organs throughout the tissue bank's or hospital's chain of custody of the materials would be beneficial. Also advantageous would be a system that reliably enables the tracing back of tissues or organs from patient to supplier after an adverse reaction is detected.

### SUMMARY OF THE INVENTION

The present invention provides a comprehensive tissue management method and system for transplant materials like tissues and organs. The tracking portion of the system verifies that staff members of a medical establishment like a hospital have handled, stored, transported, reconstituted, and used the tissue or organ materials in a safe and regulatory-compliant manner from the point of receipt to the point of issuance or surgical use throughout the hospital's organization. The tracing portion of the system creates an integral record that documents which hospital staff members have provided which processing steps to the tissue or organ, any associated materials used in conjunction with such tissue or organ, and an identification of the tissue or organ that was transplanted or implanted inside a patient, so that the tissue or organ can be reliably traced back to its donor or tissue bank source in the event of an adverse reaction by the patient after the surgery, or the tissue or organ can be traced in response to a warning received from the donor or tissue bank. Such adverse reaction investigation is preferably conducted utilizing medical cladistics to identify risk clades.

### BRIEF DESCRIPTION OF THE DRAWINGS

In the accompanying drawings:

FIG. 1 is a schematic diagram illustrating the comprehensive tissue management method of the present invention.

FIG. 2 is a schematic diagram showing the comprehensive tissue management method, storage and acquisition.

FIG. 3 is a schematic diagram showing the comprehensive tissue management method, release and utilization.

### DETAILED DESCRIPTION OF THE PREFERRED EMBODIMENT

A comprehensive method for safe management of TM transplantation is provided by the invention. Such invention enables the proper choice of vendors, logging and inspection of incoming TM, product maintenance, integrated tracking and verification of the handling, storage, and use of transplant materials in a safe and regulatory-compliant manner from the point of receipt to the point of issuance throughout the medical establishment. The invention also provides for prompt investigation of any adverse reaction suffered by a patient who receives the transplant material through a surgical procedure. Additionally, it ensures a complete documented history of the transplant material within the medical establishment so that the transplant material can be traced back to its supplier in the event that a product recall of transplant material is warranted. This allows a timely treatment of other patients receiving similar transplants. The system and method of this invention is tailored to the specific medical establishment and its handling and use of transplant materials, so as to enhance the integrity of the system.

For purposes of the present invention, “transplantable material (“TM”)” means human cells, tissue, or organs intended for implantation, transplantation, infusion, or transfer to a patient, including, but not limited to: musculoskeletal tissues like bone, tendons, fascia, ligaments, cartilage, and bioengineered bone products; skin; cardiovascular tissues like heart valves, arteries, veins, and pericardium; reproductive cells like sperm, semen, oocytes, fertilized eggs, and embryos; cellular therapies like stem cells, progenitor cells, cord blood, placental blood, chondrocytes, bone marrow, and neural cells; dura mater; breast milk; eyes; corneas; organs; islet cells; parathyroids; autologous tissue; and synthetic and xenographic tissue used as replacements for human tissue. For purposes of this application, transplantable material also includes surgical instruments, equipment, reagents, supplies, and other materials associated with the transplanting or implanting of any transplant material into a patient.

In the context of the present invention, “medical establishment” means any organization directed to the storage, research, transplantation, or implantation of transplantable materials, including but not limited to hospitals, medical clinics, surgical centers, fertility clinics, blood banks, tissue banks, organ donor banks, university and research facilities, diagnostic laboratories, and willed body programs.

As used within this application, “supplier” means any person or entity that provides a transplantable material to a medical establishment on a profit or non-profit basis, including but not limited to live or deceased organ donors, tissue banks, blood banks, fertility clinics, laboratories, and manufacturers of synthetic or bioengineered tissue or organ products.

For purposes of the present invention, “patient” means any recipient by transplantation or implantation of a transplantable material, including without limitation humans, domesticated animals like dogs, cats, and horses, and working animals like bulls and stallions.

As used within this application, “adverse reaction” means any undesirable effect or untoward outcome consequent to or reasonably related to the transplantation or implantation of transplantable material into a patient, including but not limited to disease transmission, other infectious complications like fever or wound infection attributed to the graft or positive culture of the graft at the time of use, immune rejection, and unexplained synovitis following tendon implant.

A freeze dried bone graft implanted into a human patient will be used as an exemplary transplant material for purposes of describing the comprehensive tissue management method of the present invention in this application. It is important to appreciate that any other type of transplantable material (tissues, cells or organs) or patient is covered by this application, as well.

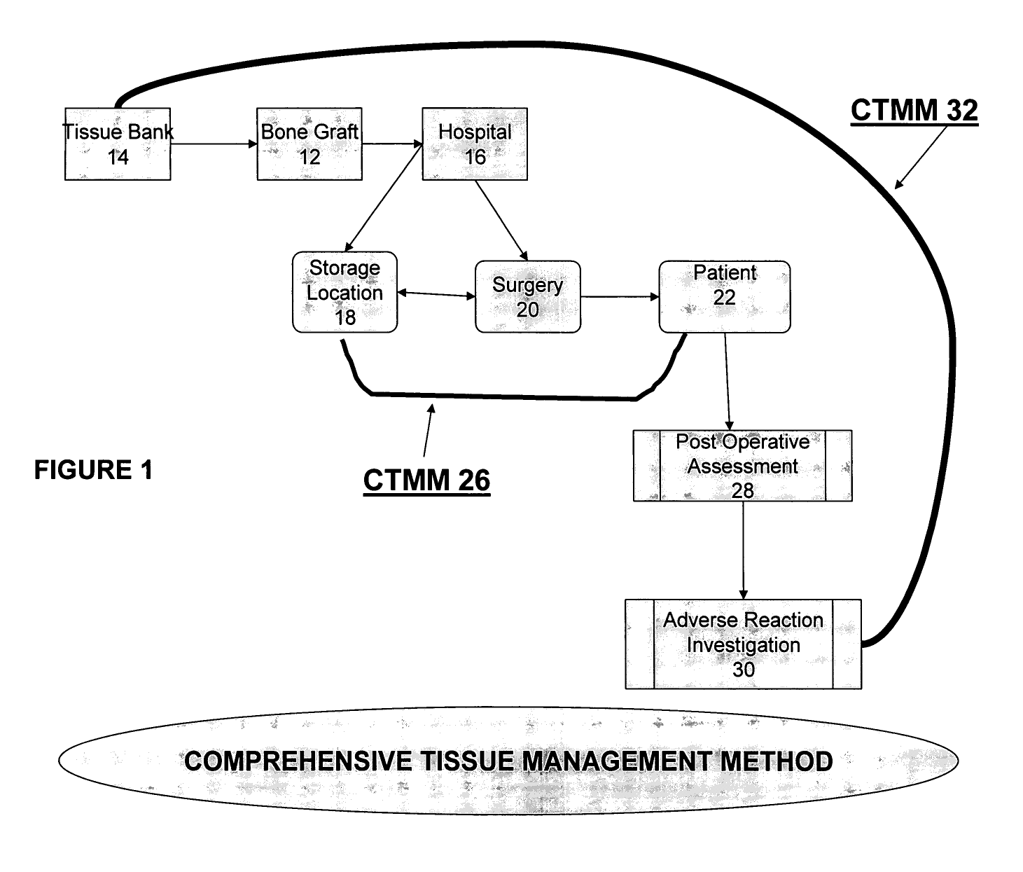

FIG. 1 shows the history of a tissue transplant from entrance into the hospital to the final implantation in a patient. When a surgeon is in need of a bone graft 12, the hospital will acquire it from a tissue bank 14. The bone graft 12 will be acquired by hospital 16 in accordance with the terms of its supply agreement with the tissue bank 14. Once the bone graft 12 is delivered to hospital 16 by tissue bank 14, it will typically be stored by the hospital. The location of storage 18 varies by hospital. It can include the blood bank, surgical department, central supply, etc. until the bone graft is needed by the hospital's surgical department 20 for implantation into patient 22. The comprehensive tissue management method portion 26 of the present invention prompts the hospital staff to undertake all of the activities necessary for the safe handling, storage, reconstitution, and implantation of the bone graft 12 in accordance with regulatory requirements and industry standards from the point of receipt by the hospital 16 until the bone graft is implanted into patient 22, and collects the necessary information to document the staff's compliance therewith.

Once the bone graft 12 has been implanted into the patient 22, the medical establishment's responsibility to the patient is not complete. Instead, the surgeon or other staff member of the hospital 16 needs to perform a post-operative assessment 28 of the patient's condition to determine whether any adverse effect like infection caused by the bone graft or surgical procedure has occurred. The hospital then notifies the tissue bank by usage of the tissue utilization information card (“TUIC”) to whom the tissue was implanted. If an adverse reaction occurs, then an investigation 30 needs to be promptly commenced to determine whether the adverse reaction was caused by the bone graft, the surgical procedure, the reagents used to reconstitute the bone graft or some independent condition of the patient 22.

The comprehensive tissue management method portions 26 and 32 of the present invention must have compiled a sufficient documentary history of the bone graft throughout its life in the hospital to enable the investigation to identify each aspect of the handling, storage, reconstitution, and surgical implantation of the bone graft 12 back to tissue bank 14 as the supplier in the event that a recall of other transplantable material from the same donor is necessary. Determination of medical treatment of other patients receiving such similar tissue products must be conducted.

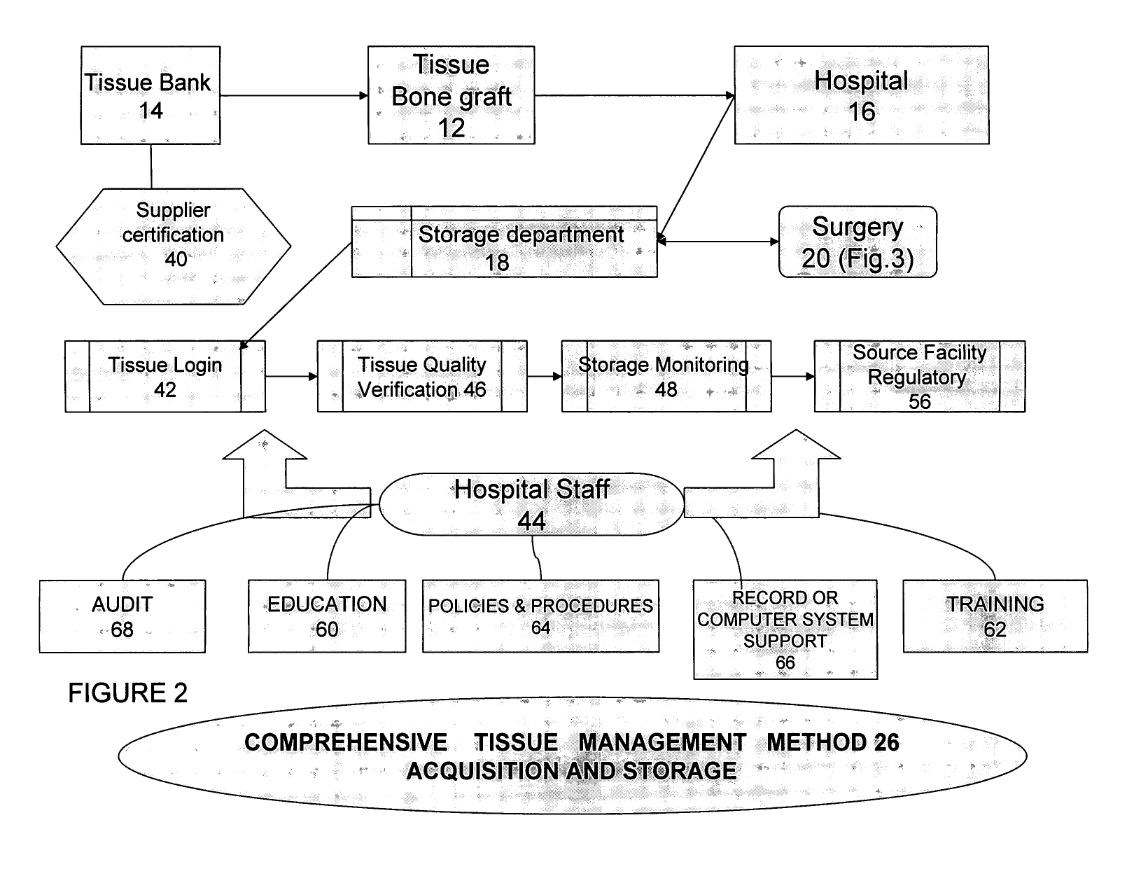

The acquisition and storage portion 26 of the comprehensive tissue management method is shown in detail in FIG. 2. The first aspect of the system is supplier certification 40 of the tissue bank 14. The present invention prompts the medical establishment to create criteria to determine the guidelines for selecting a vendor.

Rather than merely rely upon the name and reputation for quality and safety of the supplier as many hospitals do, this process step of the system requires affirmative verification that the tissue bank complies with all of the regulatory requirements and industry standards applicable to the sourcing, storage, and handling of transplant materials. At the most basic level, this can consist of documenting that the tissue bank 14 is currently registered with the FDA to supply that kind of tissue—in this case bone graft 12. Such FDA registration documentation may be obtained directly from the tissue bank 14, or from the FDA, including the FDA's Website: https://www.accessdata.fda.gov/scripts/cber/CFAppsPub/tiss/index.cfm.

Some states like New York, California, Georgia, Maryland and Florida require that tissue source facilities (processors, tissue banks, and distributors) be specially licensed for this purpose. Again, documentation of this state license may be obtained directly from tissue bank 14, or else from the applicable state licensing agency. One must ensure that the FDA registration and applicable state licensure pertain to each type of transplant material to be sourced from tissue bank 14. Supplier certification step 40 more preferably may include directly auditing the tissue bank concerning their policies and procedures for sourcing, storing, handling, and transporting the transplant materials, and personally inspecting their facilities. Much relevant information can be obtained from such direct efforts to certify the performance of the tissue bank 14.

This supplier certification step 40 can also preferably be supported by the hospital 16 entering into a memorandum of understanding or other contract with the tissue bank to comply with all regulatory requirements and industry standards for safely sourcing, storing, handling and transporting the transplant materials. The tissue bank may take its responsibilities more seriously if it risks contractual breach and damages in addition to potential loss of its FDA registration and state license.

Next, all bone grafts 12 supplied by tissue bank 14 to hospital 16 should be logged in accordance with step 42. Part of this invention's process is to determine where in the hospital this should be done, i.e. blood bank, central supply, or surgical department. This is determined by the skill of the staff, the availability of staff 24 hours a day, and the ability to monitor and maintain storage equipment and alarms. The invention requires that a specific policy and procedure be established regarding how the tissue pieces will be accepted from tissue bank 14, and which specific staff 44 will be responsible for logging in the tissue. The logging process includes a unique identification number for each piece of tissue (specially assigned where necessary), coupled with the expiration date and acceptable temperature range for each tissue product.

In some cases, tissue sent to the surgery unit 20 that was not used within the surgical procedure may be returned to the storage location 18 for use by a future patient. The login step 42 should address the login procedure for such returned tissue for re-issuance for future surgeries.

The invention requires the hospital to establish inspection criteria 46 for the incoming transplantable material. This process step includes verification upon receipt of the transplantable material product from the tissue bank 14 that package integrity of the tissue is satisfied to avoid infection or spoilage of the tissue. The staff member 44 should also verify that the temperature range for the piece of transplantable material during transport from tissue bank 14 was controlled and acceptable in accordance with FDA regulatory standards or the manufacture's recommendations.

The invention also requires the medical establishment to create specific policies and procedures regarding how it should handle, store, and transplant the transplantable material 12. This determination 48 begins with careful review and maintenance on file of the package inserts for each type of transplantable material used by the establishment. Each supplier of tissue has created different instructions for how their products should be transported, handled, stored reconstituted, and used. These instructions are approved by the FDA. Great care should be taken to ensure that the package integrity is maintained for the products during transportation and handling in order to avoid introduction of contaminants into the product.

The transplantable materials must also be maintained at their proper temperatures during their storage at ambient temperature or in refrigerators and freezers at the hospital. The FDA-approved package insert for each type of tissue product will specify the appropriate temperature or temperature range. The hospital must determine what types of refrigerator or freezer units it will use, along with the associated monitoring and alarm equipment. Appropriate policies and procedures need to be established to ensure continuous monitoring of the temperature conditions in the refrigerator and freezer units with a central alarm system or chart recorder. A number of transplantable material products that are stored at ambient temperature do not specify the temperature range that needs to be maintained. The invention requires that the hospital create an appropriate temperature range. If not specified, the invention suggests careful review of the package inserts for the ambient temperature products used by the hospital. The range is then created by taking the highest low temperature identified in the package inserts and the lowest high temperature designated in these inserts.

Alarms for each storage unit should be set so that the alarm sounds before the temperature within the unit exceeds the predetermined temperature range. In this manner, the ambient storage area, refrigerator or freezer units will maintain a temperature condition that accommodates the transplantable materials stored within.

Schedules for the periodic inspection and maintenance of the refrigerator and freezer storage units must be created. Periodic testing of the alarm systems operatively connected to the storage units and the emergency power source should also be scheduled. It is also important to maintain daily records of the actual storage unit temperature conditions to prove that the transplantable materials, in fact, were stored at their required conditions. Each piece of transplantable material should be identified within the log record by its unique identifier number, date, time and storage or handling location to document its exact condition until final disposition, transplant or discard.

If the surgery unit 20 maintains its own storage units for transplantable materials, then it must establish similar policies and procedures for monitoring the temperature conditions therein, inspecting the valid operating conditions thereof, and documenting the actual temperature conditions of the refrigerator and freezer units. The surgical unit 20, however, must also maintain precise records for each and every piece of transplantable material that it handles and uses in surgical procedures. Such records should identify the specific piece of transplantable material, its condition and all reagents or other supplies introduced to it prior to its transplantation or implantation into the patient.

Another important step carried out by staff within the surgical unit 20 will be the reconstitution of the transplantable material from its storage condition to a condition necessary for its use in surgery. This is determined by the package insert and current good medical practice. This will typically include the immersion of the tissues, like bone, into a sterile saline bath with or without antibiotics. Other human cells, tissues and organs are reconstituted with appropriate reagents. The nurse or other staff member designated to conduct such reconstitution step must carefully read and adhere to the specific instructions printed in the package insert for the transplantable material product and make the implanting surgeon aware that they are deviating from the FDA-approved package insert. The surgeon decides based on his medical judgment whether the change from the package insert is merited. At the end of the surgical procedure, the tissue usage identification cards (“TUIC”) must be completed for the product implanted into the patient and promptly returned to tissue bank 14.

In some cases, a hospital 16 may actually serve as a supplier of transplantable materials to other medical establishments. This often is the case for “sister” hospitals or large hospitals that internally source transplantable materials and use them in their other surgical procedures. The comprehensive tissue management method 26 requires the hospital 16 to periodically ask itself these questions:
Does it produce human cells, tissue and cellular and tissue-based products (“HCT/Ps”), including stem cells, reproductive cells, tissue or surgical bone?
Does it store purchased tissue and ship it to another institution that is not part of its organization? For example, does it supply a VA Medical Center or other hospital within the community?
Does it store tissue from one patient case on the chance that the surgeon needs it for another patient with the exception of vessels used in organ transplant?
Does it perform additional processing on incoming tissue? For example, gas sterilization of bone for further use.
Does its testing lab perform tissue donor testing for communicable diseases?
Does its lab test specimens for organ donors, and are such results used to determine eligibility for tissue donors?

### If the answer to any of these questions during this source facility regulatory compliance step 56 is “yes,” then the hospital 16 must register itself with the FDA much like a tissue bank 14 does.

A critical component for the comprehensive tissue management method 26 of the present invention is the hospital staff 44. A specific staff member must be designated with responsibility for each of the process steps described above. Every effort must be made to ensure that the responsible staff member has the required educational background 60 for conducting the process step. Also important is ensuring that such staff members will actually be available at the relevant time to carry out the activity step. For example, if transplantable material is maintained in a surgical unit and the responsible person unavailable to respond to an alarm sounding on a refrigerator unit this undermines proper operation of the comprehensive tissue management method 26. All designated staff members should be provided the necessary training 62 to discharge their responsibilities. They should also be provided the necessary policies and procedures 64 by hospital management that allows them to perform their duties without risk to themselves or the patient.

In order to ensure that the above-described process steps are carried out, the comprehensive tissue management method 26 should be embodied with a record-keeping system that requires the completion of individual records 66 attesting to the completion of the individual process steps. The comprehensive tissue management method is preferably embodied within a computer tissue tracking and tracing software system. Individual screen shots for a process step could require completion of the necessary data entry before the computer can progress to the next screen shot.

Finally, a periodic internal audit 68 must be conducted by the hospital 16 for all of the process steps that form the comprehensive tissue management method 26. Only in this manner can the hospital management be assured that their staff is complying with the necessary policies and procedures, and take corrective action where necessary.

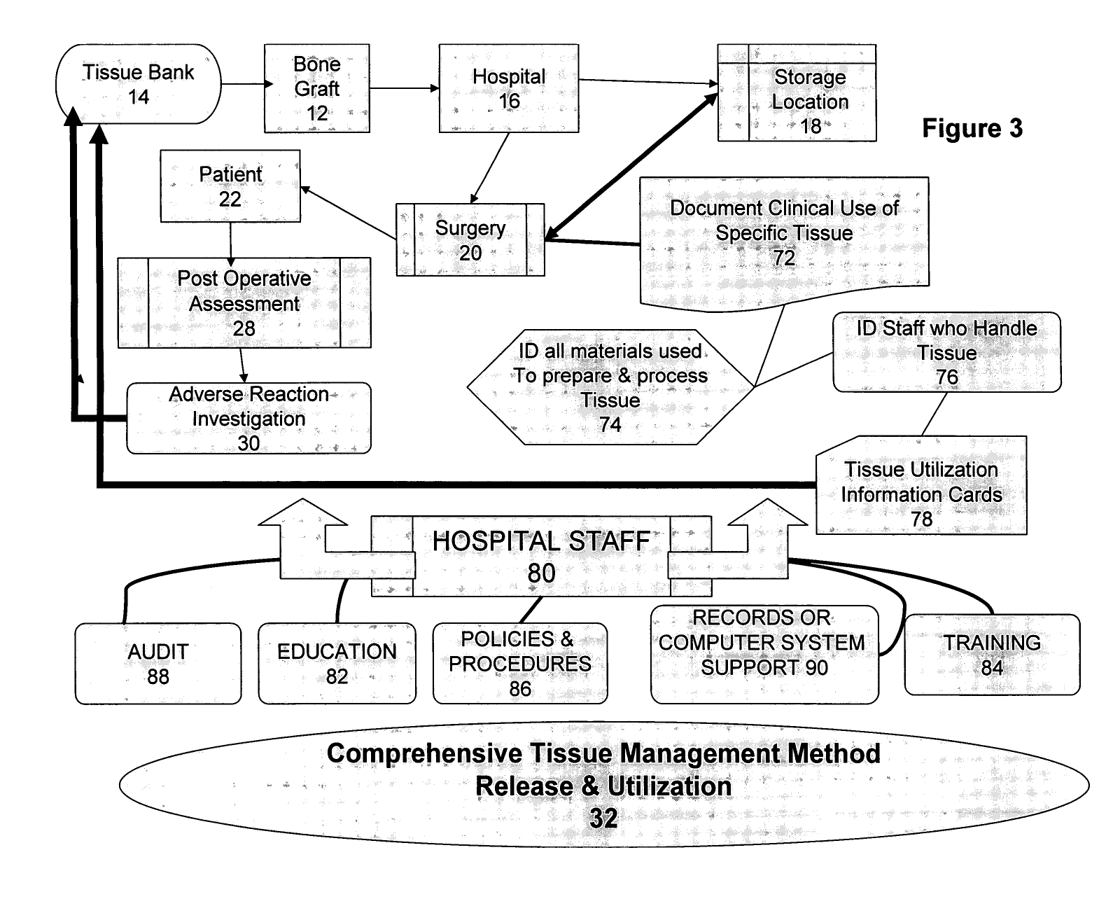

FIG. 3 illustrates the tissue tracing portion 32 of the present invention in greater detail. The objective of this system is to provide an adequate record of the transplantable material's history in case of an adverse reaction 30 in patient 22 after surgery or, if the tissue bank issues a recall and the patient who receives the transplantable material needs to be identified. For example, if the adverse reaction is an infection and is due to the operative contamination or the management of the bone graft in the hospital, then the problem is localized to the hospital and can be addressed by improving the hospital's practices. If, on the other hand, the infection is determined by investigation 30 to have been caused by the tissue graft, then the tissue needs to be traced back to tissue bank 14, so the supplier can be warned. A recall of similar transplantable material supplied by tissue bank 14 to the hospital 16 and other medical establishments may be necessary. In some cases, follow up diagnostic and medical treatment of other patients who were implant recipients of the similar transplantable material may be warranted as well.

To monitor the history of the tissue during its life in the hospital requires documentation of multiple steps starting with its entrance in the hospital and its maintenance in the storage locality 18. This will include assigning a unique identification code to the bone graft products, confirming the package integrity and safe condition of the bone graft upon receipt form the tissue bank 14, recording the actual temperature conditions, and identifying all staff members who handle the bone graft and the date and time of any such possession or handling activities from the time of initial login until the bone graft is transported to the surgical unit 20 for use in a surgery. Many of the process steps described above for the comprehensive tissue management system 26 can be employed to support the documentation steps.

Once the transplantable material 12 reaches the surgical unit 20, thorough records need to be created concerning the clinical use 72 of this specific bone graft. This includes identification of the staff member who accepts the bone graft, identification of all surgical staff members involved in preparing the bone graft for its use in the surgical procedure, and recording the dates, times and personnel for all such activities.

An important processing step by the surgical unit is the reconstitution of the bone graft product prior to its implantation in patient 22. As previously described, this should be done strictly in accordance with the instructions on the package insert. Documentation should be produced pursuant to step 74 to indicate what supplies like syringes, heparin, saline or basins were actually used to reconstitute or prepare the bone graft, and confirm that all such supplies were used prior to their indicated expiration dates.

An identification of all staff members, including the surgeon, who handle, prepares and implant the bone graft should be recorded, including the date and times of such activities in accordance with step 76. At the end of the surgical procedure, the tissue usage information card should be completed pursuant to step 78 and returned to tissue bank 14 to provide additional documentation of the clinical use and disposition of bone graft 12 in patient 22. Specific policies and procedures need to be established to determine who will complete these cards according to the tissue bank requirement, since completion and return of such tissue usage information cards is frequently overlooked within the hospital industry.

The essential element of the comprehensive tissue management method 32 of the present invention is thorough and concurrent record keeping. Failure to document important steps in the acceptance, handling, storage, transportation, processing and clinical use of the tissue and associated reagents will greatly undermine the integrity of the system and put the patient at risk. Likewise, documentation of the necessary information at a point in time substantially after the acceptance, handling, storage, transportation, processing, or surgery activity will call into question the accuracy and validity of the system.

The hospital 16 should establish the time period for retention of each record within portion 32 of the comprehensive tissue management method. In the case of login sheets and recorded procedures for the transplantable material, such records should be maintained for at least 10 years. Any records related to the transplantable material like tissue usage information cards, preparation materials, central alarm/chart recorders must be kept 10 years after the date of distribution, transplant or expiration date of the material in question, whichever is latest.

A procedure for version control must also be established for all procedures that affect the transplantable material, including a unique name for each procedure, a version number and date when it became effective, and when the previous version no longer applies in order to keep the documentary records and procedure requirements clear.

As described previously for the portion 26 of the comprehensive tissue management method, designated hospital staff members 80 need to be assigned responsibility for keeping and maintaining all records and other documents associated with the comprehensive tissue management method. While a requisite degree of education 82 may be helpful, character traits of conscientiousness and thoroughness, as well as available time may be more helpful for any such staff member. All designated staff members should also be provided adequate training 84 in such tasks as how to read temperature charts, inspect refrigeration or freezer storage units, confirm the instructions on transplantable material product insert sheets, etc. These hospital staff members 80 should also be provided understandable policies and procedures 86 to help them do their part to support the integrity and accuracy of the comprehensive tissue management method 32. Periodic internal and external audits 88 should be scheduled by hospital management to confirm that all of the necessary steps pursuant to the comprehensive tissue management method are accomplished.

A paper or computer software record keeping system 90 with adequate supporting policies and procedures must be established to ensure the integrity of the comprehensive tissue management method. Whether the process steps have been documented manually or electronically, each step much be completed and checked before the next step in the process can begin. This can include completed paper or electronic records that must be confirmed as received by hospital management before the next clinical process step for the transplantable material product can occur. Within the computer software product, a particular record-keeping screen shot can refuse to be closed before completion and transmission within the record management process. Failure to do so would prevent the staff member from progressing to his next task. The comprehensive tissue management method is predicated upon the proposition that hospital management and staff will actively investigate 30 and take corrective action in response to any adverse reaction in a patient 22. To accomplish thorough investigations of adverse reactions the invention has created the concept of “Medical Cladistics.” Cladistics is the branch of biology that determines evolutionary and taxonomic relationships between organisms based on derived similarities. Similarly, medical cladistics looks for derived similarities in investigation of clinical problems such as adverse reactions so appropriate risk categories can be created. For example, if a patient develops an infection following surgery in which tissue has been used, and an investigation shows that other patients were operated on in the same operating room (“OR”) suite developed similar infections, the “risk clade” is the OR suite, not the transplantable material, or the OR staff and the equipment sterilizer.

If a transplantable material product was associated with an infection, then the risk clade would have to be identified. Some transplantable material products can be more harshly treated than others. For example, tendons do not receive the same sterilizing treatments used by some tissue processors for bone. If an infection due to the transplantable material were bacterial and the transplantable material product implicated was a tendon, than the risk clade would be the other tendons from the same donor, not necessarily the bone products. If the infection were hepatitis than all products from the donor may be the risk clade. If similar bacterial infections were found in multiple donors, then the processing method is the risk clade, as occurred in the MMWR case cited above.

Investigation of the risk clade involves both the hospital where the transplantable material was transplanted and the transplantable material processor. Therefore, the transplantable material supplier must be notified immediately and a full investigation of the donor and processing quality control methods would be necessary to resolve the case.

The process of identifying a risk clade begins by defining an adverse reaction. This requires thorough evaluation of the actual orthopedic or other clinical and surgical practices employed by the hospital and its physicians. Factors that may influence adverse reactions include:
Physician preference for certain proprietary transplant material products and supplies. Some products carry greater risks than others.
How such products are normally prepared within the hospital.
Pre and post-operative antibiotics that are commonly used by the surgical team.
Are tissue products soaked in antibiotics prior to their implantation?
Incidence of infections in the hospital comparing allograft versus metallic devices.
Does the surgeon perform a gram stain or culture of the tissue product prior to its implantation?
Determining whether adverse reaction in a patient is based upon cultures, symptoms, or both.

The hospital 16 must establish policies and procedures to reinforce the prompt investigation and reporting of adverse reactions to answer the following questions:
Who within the hospital's management is responsible for receiving information concerning adverse events revealed during the post-operative assessment 28?
How are these events to be documented? (e.g. evaluation, report, log, etc.).
Who is responsible for determining if the adverse event was secondary to the tissue implant?
Who notifies the tissue bank and receives their evaluation and report?
Who is responsible for requesting additional information and testing, and for completing the investigation?
What other transplant materials received from the tissue bank 14 need to be traced and quarantined within the hospital 16?
All adverse event records need to be periodically reviewed for completeness.
Who will report the investigation conclusions to the clinician?

In the event that the tissue bank reports that the transplantable material was the cause of the adverse reaction, or is otherwise contaminated, then the hospital must have in place established policies and procedures for quarantining all impacted transplantable materials stored within the hospital's inventory. Such procedures encompass the ability to trace such transplantable materials, notification of the patient's physician if the transplantable material was already transplanted, maintenance of a report or log that accounts for all such transplantable material products, and an identification of a department and specific staff members responsible for doing so.

Patients who have received a transplant from donors who subsequently are found to have HIV, HTLV-I/II, viral hepatitis, or other infectious agents known to be transmittable by tissue must also be identified and informed of infectious risks. This includes donors who have donated more than one time, such as donors of reproductive tissue, stem cells, and surgical bones. When such a donor is found to have a confirmed positive test for infectious agents, then all previous donations of transplantable materials need to be identified via the comprehensive tissue management method 32, and a determination of which donations could have occurred while the donor was within the window period for this infectious agent must be made. The hospital's procedures in cooperation with the supplier should include:
Who is responsible for identifying all previous donations?
Who is responsible for determining which previous donations are at risk?
How should all subsequent recipients of the infected transplant material from this donor be identified?
How should all such recipients be notified?
Who is responsible for counseling the recipients of the infected transplant material?
Is there a policy for follow-up testing of the recipients?
Is notification of the state health department or other regulatory agencies required?
Is there a procedure and log to document all of this information?
Is the individual who counsels the recipients properly educated and trained?

In its simplest embodiment, the system for enabling the comprehensive tissue management method of the present invention may be a paper record keeping system. This may be particularly appropriate for relatively small medical establishments that do not handle and process enough transplantable materials to justify the cost of a computer-based system. The requirements established by the medical establishment for filling our and completing such paper forms and submitting them to appropriate managers may be sufficient for ensuring that the various processing steps for handling, storage, reconstitution, and surgical use of the transplantable material by staff members are being conducted in a manner compliant with prevailing regulations and industry standards, and that a thorough document record is produced therefore. However, it is important to incorporate within such policies and procedures safeguards for ensuring that all of these internal requirements are met by staff members in a compliant and timely manner. This could include a requirement that records be filled out from one processing step before the next processing step for the transplantable material can be conducted by the same or another staff member. Periodic internal and external audits will also be important for assuring compliance.

In another embodiment of the present invention, the comprehensive tissue management system is at least partially a software program residing or interacting with hardware such as a computer system. The software program may be designed to be an expression of an organized set of instructions in a coded language. These instructions are programmed to facilitate the monitoring of the transplantable material from at least the point of sourcing, handling, transportation, storage, reconstitution and/or surgical implantation. The instructions may include the entry, reporting and/or reading of data relating to time, date, location, temperature, condition of the tissue, and/or any other information needed for monitoring the transplantable material.

The computer system on which the system resides may be a standard PC, laptop, mainframe, handheld wireless device, or any automated data processing equipment capable of running software for monitoring the progress of the transplantable material. The computer system may include a central processing unit (CPU), a memory, clock circuitry, a printer device, a display, a data interface, a network controller, an internal bus and one or more input devices such as, for example, a keyboard and/or mouse.

The CPU controls the computer system and is capable of running the system stored in memory. The memory may include, for example, internal memory such RAM and/or ROM, external memory such as CD-ROMs, DVDs, flash drives, or any currently existing or future data storage means. The clock circuit may include any type of circuitry capable of generating information indicating the present time and/or date. The clock circuitry may also be capable of being programmed to count down a predetermined or set amount of time. This may be particularly important if a particular type of tissue needs to be refrigerated or implanted in a predetermined amount of time.

The data interface allows for communication between one or more networks which may be a LAN (local area network), WAN (wide area network), or any type of network that links each party handling the tissue. Different computer systems such as, for example, a laptop and a wireless device typically use different protocols (i.e., different languages). To allow the disparate devices to communicate, the data interface may include or interact with a data conversion program or device to exchange the data. The data interface may also allow disparate devices to communicate through a Public Switched Telephone Network (PSTN), the Internet, and private or semi-private networks.

The tissue management system may include a graphic user interface (GUI) that is displayed to a user in a text or graphical form for data entry. The GUI can also be used to display the status of the transplantable material to any or selected staff members participating in monitoring the transplantable material. Additionally, the comprehensive tissue management system, computer system, and GUI can be connected to external devices such as refrigeration units to send a current temperature reading or to notify those monitoring the tissue that the temperature of the refrigeration unit has decreased or increased beyond a predetermined range. The comprehensive tissue management system, computer system, and GUI can be connected to any type of device that needs to be monitored.

The computer system can also notify those monitoring the tissue of any of the steps described above by visual and/or audible means. Continual electronic monitoring of the transplantable material allows the computer system to store and display the entire history of a particular transplantable material sample. The continual monitoring reduces the risks that the transplantable material sample's integrity has been compromised.

Adequate policies and procedures adopted by the medical establishment can help with assuring that staff members are using the computer-based system in a compliant and timely manner. Periodic internal and external audits will also be helpful.

The above specifications and drawings provide a complete description of the structure and operation of the comprehensive tissue management method for acquisition and storage of transplantable material 26 and comprehensive tissue management method for release and utilization of transplantable material 32 under the present invention. However, the invention is capable of use in various other combinations, modifications, embodiments, and environments without departing from the spirit and scope of the invention. Therefore, the description is not intended to limit the invention to the particular form disclosed, and the invention resides in the claim and hereinafter appended.

We claim:

1. A method for tracking the internal processing by a medical establishment of a piece of transplantable material provided by a supplier for subsequent transplantation or implantation into a patient, such method comprising:
(a) assigning a unique identification code to the transplantable material upon its receipt by the medical establishment from the supplier;
(b) inspecting the transplantable material upon its receipt from the supplier for an unsafe condition.
(c) ensuring that each processing step comprising handling, storage, reconstitution or surgical use of the transplantable material by staff members of the medical establishment is conducted strictly in a manner compliant with prevailing regulations and industry standards;
(d) assessing the patient after transplantation or implantation of the transplantable material for evidence of an adverse effect;
(e) investigating any such adverse effect discovered in the patient to determine whether it constitutes an adverse reaction; and
(f) reporting any such adverse reaction to the supplier that supplied the transplant material to the medical establishment.

2. The transplantable material tracking method of claim 1 further comprising designating one or more staff members of the medical establishment with specific responsibility for conducting each of the steps (a-f).

3. The transplantable material tracking method of claim 1 further comprising the step of certifying that the supplier currently possesses all required regulatory certifications and licenses to supply the transplantable material

4. The transplantable material tracking method of claim 1 further comprising evaluating the safety or health risks posed by transplantable materials supplied by the supplier against the benefits provided by such transplantable materials.

5. The transplantable material tracking method of claim 4, wherein input from a transplant surgeon is obtained when conducting the risk-benefit analysis.

6. The transplantable material tracking method of claim 1 further comprising ensuring that a staff member of the medical establishment who performs any of the steps (a-f) possesses a relevant educational background for performing such step.

7. The transplantable material tracking method of claim 1 further comprising providing relevant training to a staff member of the medical establishment who performs any of the steps (a-f).

8. The transplantable material tracking method of claim 1 further comprising providing relevant policies or procedures in support of the reliable conduct of any of the steps (a-f).

9. The transplantable material tracking method of claim 1 further comprising periodic auditing of the medical establishment to ensure that each step (a-f) is being conducted properly.

10. The method for tracing a piece of transplantable material received by a medical establishment from a supplier throughout the internal life of the transplantable material within the medical establishment until it is transplanted or implanted into a patient, such method comprising:
(a) assigning a unique identification code for the transplantable material upon its receipt by the medical establishment from the supplier;
(b) documenting each processing step comprising login, handling, storage, reconstitution, or surgical use applied by staff members to the transplantable material;
(c) documenting the identity of each staff member who comes into contact with the transplantable material;
(d) documenting the date and time of each processing step applied to the transplantable material;
(e) documenting all materials used to prepare and process the transplantable material;
(f) documenting the unique identification code of the transplantable material transplanted or implanted into the patient; and
(g) tracing the transplantable material back to the supplier that supplied it to the medical establishment or to the specific processing step applied by a staff member of the medical establishment to the transplantable.

11. The transplantable material tracing method of claim 10 further comprising designating one or more staff members of the medical establishment with specific responsibility for conducting each of the steps (a-g).

12. The transplantable material tracing method of claim 10 further comprising sending to the supplier a tissue utilization information card for the transplantable material transplanted or implanted to the patient.

13. The transplantable material tracing method of claim 10, wherein the tracing step is conducted in response to an adverse reaction detected in the patient into whom the transplantable material was transplanted or implanted.

14. The transplantable material tracing method of claim 13, wherein the supplier that supplied the transplantable material to the medical establishment is notified of the adverse reaction in the patient.

15. The transplantable material tracing system of claim 10, wherein the tracing step is conducted in response to a warning received from the supplier that supplied the transplantable material to the medical establishment.

16. The transplantable material tracing system of claim 13, wherein the adverse reaction investigations is conducted utilizing medical cladistics to identify risk clades.

17. The transplantable material tracing method of claim 10 further comprising ensuring that a staff member of the medical establishment who performs any of the steps (a-g) possesses a relevant educational background for performing such step.

18. The transplantable material tracing method of claim 10 further comprising providing relevant training to a staff member of the medical establishment who performs any of the steps (a-g).

19. The transplantable material tracing method of claim 10 further comprising providing relevant policies or procedures in support of the reliable conduct of any of the steps (a-g).

20. The transplantable material tracing method of claim 10 further comprising periodic auditing of the medical establishment to ensure that each step (a-g) is being conducted properly.
